(19) United States
(12) Patent Application Publication
(10) Pub. No.: US 2006/0093675 A1
(43) Pub. Date: May 4, 2006

(54) Intravaginal treatment of vaginal infections with metronidazole compositions

(76) Inventors: Mathew Ebmeier, Fort Collins, CO (US); Robert Lathrop, Fort Collins, CO (US)

Correspondence Address: SCHWEGMAN, LUNDBERG, WOESSNER & KLUTH, 1600 TCF TOWER, 121 SOUTH EIGHT STREET, MINNEAPOLIS, MN, 55402, US

(21) Appl. No.: 10/978,235

(22) Filed: Oct. 29, 2004

(51) Int. Cl. A61K 9/14; A61K 31/4164

(52) U.S. Cl. 424/487; 424/488; 514/398

(57) ABSTRACT

The present invention provides a buffered non-flowing composition suitable for the treatment of bacterial vaginosis. The composition includes metronidazole in a concentration of about 0.50% (w/w) to about 1.50% (w/w). The metronidazole is present together with a buffer system in a physiologically tolerable medium. The buffer system provides an acidic buffered pH value for the composition in the range of about 5.0 to about 6.0. The present invention also provides for a method for inhibiting a microorganism. The method includes contacting a microorganism with an effective amount of the composition of the present invention, for a period of time effective to inhibit the microorganism. The present invention also provides for a method for treating bacterial vaginosis in a human patient. The method includes intravaginal administration to a patient in need of such treatment an effective amount of the composition the present invention. The composition is introduced into the vagina at least once a day for a time period of at least one day.

### BACKGROUND OF THE INVENTION

Bacterial vaginosis (BV) is associated with an increased volume of vaginal discharge which has a foul, fishy odor. Vaginal pH is elevated from the normal range (pH 3-4) to values ≧pH 4.7. The odor and elevated pH are caused by a high level of amines, most notably trimethylamine, in the vagina. These amines are volatilized when the pH is raised, for example, as with addition of KOH or interaction with semen. The vaginal discharge is homogenous in appearance as opposed to the flocculent discharge seen in Candida vaginitis. In contrast to candidiasis and trichomoniasis, itching generally is not associated with BV. A microscopic examination of a wet mount of the vaginal discharge in BV reveals an absence of polymorphonuclear leukocytes (PMNs). In contrast, the presence of many PMNs in a vaginal discharge is indicative of trichomoniasis, gonorrhea, or chlamydial cervicitis.

The causative organism for BV is a matter of some controversy. Gardnerella vaginalis is usually implicated as the causative agent because it is isolated from 98% of women with BV. However, G. vaginalis is also recovered in smaller numbers as normal flora in the vagina of asymptomatic women in incidences as high as 68% (Totten et al, 1982).

In those conditions where Gardnerella is present in higher concentrations, there is a significant decrease in the numbers of Lactobacilli present compared to the normal vagina. The normal vaginal flora is composed predominantly of Lactobacillus species, with an average pH of 4.0 (Hill and Embil, 1986; Bartlett and Polk, 1984). This low pH fosters growth and maintenance of the acidophilic Lactobacilli (anaerobic and facultatively anaerobic Gram-positive bacilli) that dominate the normal flora in concentrations of 10⁸ to 10⁹ Lactobacilli per milliliter of vagina secretions (Larsen and Galask, 1982; Rein, 1985). While it is not known if a decrease in the Lactobacilli allows the Gardnerella to multiply, or if the increased numbers of Gardnerella actually inhibit the Lactobacilli, it is postulated that hydrogen peroxide production by certain Lactobacillus species represents a mechanism by which Lactobacilli regulate the growth of other organisms in the vagina (Eschenbach et al., 1989). In any event, if the predominant microorganism present in the wet mount is not Lactobacilli, then BV must be suspected.

There have been overgrowths of other microorganisms seen in BV. Mycoplasma hominis and anaerobic bacteria including Bacteroides, Peptococcus, and Mobiluncus are also highly associated with BV (Eschenbach et al, 1988). In BV, G. vaginalis and the anaerobes can be present in overgrowths 1000 to 100,000 times more frequently than normal. It is also not known if the anaerobes are a result of the decreased amounts of Lactobacilli, or if they are responsible for the decrease. These organisms are present, however, in concentrations that should be considered pathogenic (Mead et al, 1986).

Characteristically seen in the wet mount in BV are abnormal cells termed “clue cells.” These clue cells are vaginal epithelial cells with such a heavy coating of bacteria surrounding them that their peripheral borders are obscured (Eschenbach et al. 1988).

Peeters and Piot (1985) developed an experimental model of the G. vaginalis adherence to vaginal epithelial cells forming “clue cells.” Using this model they found that the optimum pH for adhesion in vitro was pH 5 to 6 (the vaginal pH of women with bacterial vaginosis) and adhesion was limited at pH 3 to 4 which is the normal pH of vaginal fluid in women without vaginosis. If the same is true in vivo, a rise in vaginal pH is possibly a prerequisite in the pathogenesis of BV and perhaps precedes the formation of the pathognomonic “clue cells.”

The antibacterial activity of Lactobacilli against other microorganisms has been suggested (Mardh and Soltesy, 1983). Skavin and Sylwan (1986) found that Lactobacilli strains inhibited growth of bacterial strains implicated in and isolated from women with BV in in vitro cultures. The bacterial strains tested included Mobiluncus mulieris, Mobiluncus curtisii, G. vaqinalis, Peptococcus species, Peptococcus asaccharolvticus, Peptostrepotococcus anaerobius, Grampositive anaerobic coccus, and Bacteroides species. They also found that the lowest pH which would allow macroscopically visible growth of these bacterial strains ranged from pH 5.0 to 5.5. This data supports the importance of establishing and maintaining the presence of the Lactobacillus-dominated normal vaginal flora and the necessary pH environment for their growth and inhibition of other BV associated bacteria.

A clinical diagnosis of BV is made if three or more of the following four clinical criteria are present: (1) a homogenous discharge; (2) a pH.gtoreq.4.7; (3) a “fishy” amine odor upon the addition of 10% KOH to discharge; (4) presence of epithelial clue cells representing greater than or equal to 20% of vaginal epithelial cells (Eschenbach et al, 1988).

The efficacy of metronidazole in the treatment of BV is known. A marked effectiveness for metronidazole, given at 500 mg by mouth, twice daily for seven days has been demonstrated. Cure rates of 80-90% have repeatedly been reported since that time by the oral route of administration (Pheiffer et al., 1978; Balsdon et al., 1980; Eschenbach et al., 1983; Purdon et al., 1984; Charles et al., 1985; Swedberg et al., 1985; Malouf et al., 1981; Amsel et al., 1982; Hagstrom and Lindstedt, 1983; Mead et al., 1986). These studies employed the oral use of metronidazole in doses that ranged from 400 to 500 mg twice daily for three to seven days or 2 grams in a single dose. Heretofore, it has been generally accepted that the oral administration of metronidazole for five to seven days is the most effective way to treat DV; however, such a treatment for BV is not approved by the United States Food and Drug Administration (FDA). The Center for Disease Control recommends a dose of 500 mg of metronidazole given twice daily for seven days for treatment of bacterial vaginosis (CDC, 1985).

The adverse reactions from oral administration of metronidazole can be extensive, however. For metronidazole, the “Modern Drug Encyclopedia”>A. J. Lewis, Editor, pub. by Vocke Medical Books, New York, N.Y. (1979), contains the following statement on metronidazole: “Adverse Reactions: Nausea, headache, anorexia, vomiting, diarrhea, epigastric distress, abdominal cramping, constipation, a metallic, sharp and unpleasant taste, furry tongue, glossitis, stomatitis, leukopenia, dizziness, vertigo, incoordination, ataxia, convulsive seizures, numbness or paresthesia of extremities, fleeting joint pains, confusion, irritability, depression, insomnia, mild erythematous eruption, weakness, urticaria, flushing, dryness of the mouth, vagina or vulva, pruritus, dysuria, cystitis, sense of pelvic pressure, dyspareunia, fever, polyuria, incontinence, decrease of libido, nasal congestion, proctitis, pyuria, and rarely, an unexplained darkening in the color of the urine have been reported. Flattening of the T wave may be seen in electrocardiographic tracings.”

The need for providing safe and effective treatment for BV (without, for example, the side effects associated with the oral usage of metronidazole) assumes a more acute and pressing status when epidemiological trends and possible sequelae of a serious nature are given consideration. For example, vaginal infection with G. vaginalis, has been associated with possible sequelae, such as pelvic inflammatory disease, endometritis, and premature labor (Mead et al., 1986) that have an attendant, significant morbidity profile. Although there is no direct evidence linking BV with these conditions, it is not unreasonable to assume that an overgrowth of 10,000 to 100,000 anaerobic organisms in the vagina may result in certain genital diseases (Mead et al, 1986). Moreover, in the last decade there has been a tendency towards a reduction in gonorrhea and trichomoniasis while, during the same time span, there has been an increase in the so called “non-specific genital disease” (Staerfelt et al, 1983). Further, BV may account for significantly more total vaginitis patients than either Candida or trichomoniasis (Mead et al, 1986).

Since BV is a localized problem, intravaginal application of metronidazole should in principle be clinically effective. Moreover, since in intravaginal application, unaffected organ systems would be subjected to significantly lower or non-detectable levels of metronidazole, its side effects would be therefore minimized or eliminated.

A desirable treatment for BV would be an intravaginal composition that delivers a minimum effective dose of metronidazole while it simultaneously adjusts and maintains the vaginal pH at about the normal physiological range while promoting the growth of Lactobacillus species that produce hydrogen peroxides and controlling the overgrowth by pathogens.

Intravaginal metronidazole therapy for BV has been studied (Bistoletti et al., 1986). The authors compared oral treatment which consisted of 400 mg of metronidazole in the morning and evening for seven days to vaginal treatment consisting of the application of a vaginal insert containing 500 mg of the drug every evening for seven days. Thus, the total dose given was 5.6 g in the oral, and 3.5 g in the vaginal, treatment groups. The findings in the 38 patients who completed the study showed a cure rate, at four weeks after initiation of therapy, to be 15 out of 19 (79%) for the vaginal treatment group and 14 out of 19 (74%) after oral treatment. Cure was based on assessment of pH, vaginal discharge, the 10% KOH amine test, and examination of a wet smear for clue cells. These same authors also reported that lactate-producing microorganisms (Lactobacilli and aerobic Streptococci) were found more frequently after vaginal than after oral treatment and speculated that this difference may be due to the higher local concentration of the drug achieved by intravaginal administration. In this regard, a low concentration of metronidazole has been found in the vaginal fluid after a single oral dose of 2 grams metronidazole (Davis et al., 1984). These authors concluded that topical administration of metronidazole might be more effective in re-establishing the normal microflora in the vagina. No side effects were reported related to the intravaginal use of metronidazole as the 500 mg insert. Although this study showed effectiveness of vaginally administered metronidazole, these researchers still used a relatively high dose (3.5 grams) and made no attempt to adjust and control vaginal pH. Moreover, these authors did not recognize the criticality of low pH for selectively promoting the growth of hydrogen peroxide producing Lactobacillus species.

Intravaginal sponges containing metronidazole also have been described. Brenner et al., Adv. Contracept. 2:363-368 (1986), describe the use of metronidazole and nonoxynol-9 containing sponges where each sponge contains 250 milligrams of metronidazole and 650 of nonoxynol-9 and estimate that about 160 milligrams of metronidazole in each sponge is released over a 24-hour use period.

Because of low water solubility of metronidazole, various oil-based metronidazole compositions have been developed, which are generally either creams (oil in water emulsions) or ointments (petroleum jelly based compositions) with metronidazole being dissolved/suspended in the oil/water phases.

Romanian Patent No. 80,363, published Nov. 30, 1982 (reported also at C.A. 101:116743c), describes a vaginal gel with antibiotic and anti-inflammatory activity. This gel comprises metronidazole, nystatin with other antibacterials selected from nitrofural, chloramphenicol, and tetracycline and camazulene or hexoestrol acetate incorporated into Carbopol 940®, an aqueous gel-forming polyacrylic acid polymer available from B. F. Goodrich, Cincinnati, Ohio.

Such gel formulation suffers from the disadvantage that it includes, in addition to metronidazole, various active antibiotic, antimicrobial and antimycotic agents. Such gel formulation then operates intravaginally on a broad spectrum “shot gun” basis to destroy not only the harmful bacteria associated with “vaginitis,” but also the desirable bacteria, such as the Lactobacilli and other lactate-producing organisms (e.g., aerobic Streptococci) that are present in the normal vagina. In addition, the Romanian patent teaches a gel formulation for intravaginal use which is formulated at a pH of 6 to 6.5. Hence, use of such a vaginal gel formulation is open to question from the standpoint of being a safe treatment for BV since it leaves the treated vagina in an abnormal condition where reinfection or infection by other opportunistic microorganisms are possible sequelae.

A known commercial vaginal formulation of metronidazole currently on the international market for use as a trichomonacide, but not in the United States, is produced by Rhone-Poulenc Pharma Inc. of Montreal, P.Q., Canada. This formulation is a cream which contains 500 mg of metronidazole per application (5 grams). The recommended dose for trichomoniasis is one application once or twice daily for 10 to 20 days. Therefore, the total dose recommended ranges between 5 grams and 20 grams of metronidazole. The pH value of this formulation was tested by an independent laboratory to be pH 6.1.

U.S. Pat. Nos. 5,840,744 and 5,536,743 disclose buffered non-flowing composition that includes metronidazole. The compositions are suitable for the treatment of bacterial vaginosis. The buffer system provides an acidic buffered pH value for the composition in the range of about 3.75 to about 4.25.

The need for a safe and effective treatment for bacterial vaginosis which can eliminate the invading organisms at a low, safe dose and provide the necessary vaginal environment for growth and maintenance of lactate-producing organisms without overgrowth of potential pathogens remains.

### SUMMARY OF THE INVENTION

The present invention provides a non-flowing composition suitable for the treatment of bacterial vaginosis. Compared to commercially available Metronidazole Gel formulations, the composition of the present invention includes less gelling agent (e.g., carbomer). As such, the composition of the present invention is easier to manufacture, and is less expensive to manufacture, compared to commercially available Metronidazole Gel formulations. Additionally, compared to commercially available Metronidazole Gel formulations, the composition of the present invention has a slightly higher pH value, both neat and upon dilution. Some physicians and patients may prefer a product having a more neutral pH, whether the product is neat or is diluted (e.g., 10:1 water-product).

The present invention provides a non-flowing composition suitable for the treatment of bacterial vaginosis. The composition includes metronidazole in a concentration of about 0.50% (w/w) to about 1.50% (w/w). The metronidazole is present together with a system in a physiologically tolerable medium. The system provides an acidic pH value for the composition in the range of about 5.0 to about 6.0.

The present invention also provides a gel composition. The gel composition includes: (a) an antibiotic agent, an antifungal agent, or a combination thereof; (b) a base; (c) a gelling agent; and (d) a solvent. The composition has a pH of about 5.0 to about 6.0.

The present invention also provides a gel composition. The gel composition includes: (a) an antibiotic agent, an antifungal agent, or a combination thereof; (b) a humectant; (c) a preservative; (d) a chelator; (e) a base; (f) a gelling agent; and (g) a solvent. The composition has a pH of about 5.50.

The present invention also provides a gel composition. The gel composition consists essentially of: (a) an antibiotic agent, an antifungal agent, or a combination thereof; (b) a humectant; (c) a preservative; (d) a chelator; (e) a base; (f) a gelling agent; and (g) a solvent. The composition has a pH of about 5.50.

The present invention also provides a gel composition. The gel composition includes: (a) metronidazole; (b) propylene glycol; (c) methylparaben; (d) propylparaben; (e) edetate disodium; (f) sodium hydroxide; (g) carbomer 934P; and (h) water. The composition has a pH of about 5.50.

The present invention also provides a gel composition. The gel composition consists essentially of: (a) metronidazole, (b) propylene glycol; (c) methylparaben; (d) propylparaben; (e) edetate disodium; (f) sodium hydroxide; (g) carbomer 934P; and (h) water in about 93.85% (w/w). The composition has a pH of about 5.50.

The present invention also provides a gel composition. The gel composition includes: (a) metronidazole in about 0.75% (w/w), (b) propylene glycol in about 3.0% (w/w); (c) methylparaben in about 0.08% (w/w); (d) propylparaben in about 0.02% (w/w); (e) edetate disodium in about 0.05% (w/w); (f) sodium hydroxide in about 0.25% (w/w); (g) carbomer 934P in about 2.0% (w/w); and (h) water in about 93.85% (w/w). The composition has a pH of about 5.50.

The present invention also provides a gel composition. The gel composition consists essentially of: (a) metronidazole in about 0.75% (w/w), (b) propylene glycol in about 3.0% (w/w); (c) methylparaben in about 0.08% (w/w); (d) propylparaben in about 0.02% (w/w); (e) edetate disodium in about 0.05% (w/w); (f) sodium hydroxide in about 0.25% (w/w); (g) carbomer 934P in about 2.0% (w/w); and (h) water in about 93.85% (w/w). The composition has a pH of about 5.50.

The present invention also provides for a method for inhibiting a microorganism. The method includes contacting a microorganism with an effective amount of the composition of the present invention, for a period of time effective to inhibit the microorganism.

The present invention also provides for a method for treating bacterial vaginosis in a human patient. The method includes intravaginal administration to a patient in need of such treatment an effective amount of the composition the present invention. The composition is introduced into the vagina at least once a day for a time period of at least one day.

### DETAILED DESCRIPTION OF THE INVENTION

While this invention is susceptible to embodiment in many different forms, preferred embodiments of the invention are described herein below in detail. It should be understood, however, that the present disclosure and the embodiments described herein are to be considered as exemplifications of the principles of this invention and are not intended to otherwise limit the invention, as defined by the claims herein.

The present invention is practiced therapeutically by introducing into such an afflicted vagina a therapeutically effective amount of a formulation of metronidazole, such as herein below described and exemplified. Moreover, the present invention also contemplates the use of the herein described metronidazole compositions for preventing bacterial vaginosis in human female patients that are susceptible to it. To that end, a prophylactic amount of a non-flowing, viscid composition which contains metronidazole as the sole active ingredient and has a pH value in the range of about 5.0 to about 6.0 is administered intravaginally chronically or for a time period while the susceptibility exists.

The term “vagina” as used herein is intended to be inclusive of the vaginal region generally, including also the vulva and the cervix. Also, the term “afflicted vagina” as used herein is intended to be inclusive of bacterial vaginosis (BV).

The quantity of metronidazole introduced intravaginally as a single or unit dose can vary widely, depending upon many variables, such as the age and physical condition of the patient, the extent of the patient's affliction, the frequency of administration, the need for prophylaxis, and the like.

The term “unit dose” or “unit dosage form” as used herein refers to physically discrete units of such composition suitable for use as unitary dosages by human female subjects. Each unit contains a predetermined quantity of metronidazole calculated to produce the desired therapeutic effect in association with the required pharmaceutical vehicle. The exact novel unit dosage form(s) of the invention to be used for any given patient is/are dictated by, and directly dependent on (a) the unique characteristics of the metronidazole compositions and the particular therapeutic effects to be achieved, and (b) the characteristics, especially the release rate of metronidazole from the particular composition contemplated for the intended therapeutic use, as disclosed in detail in the present specification, these being features of the present invention.

An article of manufacture embodying the present invention typically includes a packaging material and contained therein a pharmaceutical agent consisting essentially of metronidazole and the aforementioned system in a physiologically tolerable medium. The packaging material includes a label which indicates that the pharmaceutical agent can be used for ameliorating the symptoms of bacterial vaginosis, preferably by administering about 37.5 milligrams of metronidazole in an aqueous gel twice daily for five days.

Any convenient non-flowing, i.e., self-supporting and viscid, such as gel, paste, cream, and the like, unit dose form can be employed in practicing this invention. A presently preferred technique is to extrude a non-flowing composition, such as a gel composition, through a tubular applicator from a storage vessel, such as a syringe, squeezable tube, or the like, into the afflicted vagina. The volume of gel composition so contained within a single such vessel is conveniently and preferably selected so as to constitute a single dose, or two doses, or the like, so as to facilitate administration of a desired controlled dose to a patient. The storage vessel is initially sealed, but is opened at the time of use. If more than a single dose is present, the vessel is preferably resealable by a suitable closure means.

Another presently preferred technique is to employ a single use packet (such as a small envelope-like structure, or the like) containing an intended single unit dose. The packet is initially sealed, but is opened at the time of use by tearing, cutting, or the like at a desired or planned location in the packet after which the packet is manually squeezed so that the contents are directly administrable as desired.

The quantity of metronidazole contained in a unit dose is generally at least about 20 milligrams (mg), and is not more than about 100 mg. A typical and presently preferred unit dose in a gel vehicle is in the range of about 20 to about 40 mg, most preferably about 37.5 mg, per dose.

Such a quantity can be administered one to three times daily (that is, at spaced intervals in a 24 hour period) in a single day or over a period of up to ten days. The total daily dose thus delivered can range from about 20 to about 100 mg. In a gel form of the composition, a daily dose in the range of about 30 to about 80 mg usually is sufficient. The usual total dose during the course of therapy for compositions of the present invention is in the range of about 100 mg to about 375 mg. A presently preferred administration procedure is to employ a unit dose of 5 grams of gel (delivering a dose of 37.5 mg of metronidazole) administered once or twice daily for a period of about five days, thereby to deliver a total dose in the range of about 185 mg to about 375 mg. Those skilled in the art will appreciate that the foregoing dose levels are provided illustratively, and that higher and lower dose levels can be employed without departing from the spirit and scope of the present invention.

Such doses are significantly lower than the comparable 7 gram dose (500 mg b.i.d. employed for 7 days, the standard BV dosage) as currently utilized and recommended by CDC. The low daily dose of the particularly preferred gel composition directly applied to the site of activity decreases the risks of dose related side effects and potential systemic activity. The effectiveness of this novel, low dose therapy is believed to be related to the combination of site specificity, controlled release, pH adjustment, control of vaginal environment, and provision for reestablishment of necessary normal vaginal flora, i.e., lactate producing microorganisms and hydrogen-peroxide producing microorganisms.

For prophylactic purposes, the amount of metronidazole administered is in the range of about 20 milligrams to about 80 milligrams, more preferably in the range of about 30 to about 40 milligrams per dose. These prophylactic amounts can be introduced intravaginally as a single dose or more than one dose, as desired, preferably twice a week on non-consecutive days.

The active ingredient in the present composition is 1-(2-hydroxyethyl)-2-methyl-5-nitroimidazole (metronidazole). This drug is described in U.S. Pat. No. 2,944,061 to Jacob et al., and is commercially available.

The term “metronidazole” as used in this specification and claims includes not only 1-(2-hydroxyethyl)-2-methyl-5-nitroimidazole, but also those analogs and derivatives of metronidazole (salts, esters, etc.) which are soluble in the aqueous or oil phases of the compositions described herein and which exhibit therapeutic activity when applied as taught by the present invention. A physiologically tolerable medium is utilized as the delivery vehicle for metronidazole.

The term “physiologically tolerable medium” as used herein refers to one or more viscous-to-solid materials, i.e., of non-flowing consistency, which are non-irritating to the vaginal region. While a given such medium in a presently contemplated composition can be comprised of a single material, a plurality of components can comprise such a medium as well. Examples of components include water, oil, surfactants, preservatives, penetration enhancers, preservatives, and the like, such as hereinbelow described and illustrated. For purposes of avoiding problems of pooling and running, the physiologically tolerable medium is preferably characterized by a viscosity at ambient conditions (e.g., 25° C., 760 mm Hg) with said metronidazole and also said system dissolved and/or dispersed therein which is at least sufficient to maintain a product composition of this invention in a non-flowing state.

A pH measurement can typically be made either chemically (litmus paper) or potentiometrically (pH electrode and meter). Further, a pH measurement can typically be made by either direct measurement in the finished product (non-diluted), or by an apparent measurement in the finished product diluted with purified water. The ratio of dilution between purified water and finished product may range from 1:1 to 20:1. For the compositions of the present invention, the pH measurement are based upon the sample being dilute in purified water at 10:1 dilution. This is done because there can be discrepancies with pH measurements of a sample, depending on whether the pH is measured neat or diluted, and there may be difficulties in obtaining an accurate pH measurement upon neat samples.

The compositions of the present invention can optionally be buffered.

The term “buffer system” or “buffer” as used herein has reference to a solute agent or agents which, when in water solution, stabilize such solution against a major change in pH (or hydrogen ion concentration) when acids or bases are added thereto. Solute agent or agents which are thus responsible for a resistance to change in pH from a starting buffered pH value in the range above indicated are well known. Virtually any pharmaceutically acceptable buffer system can be used which will achieve a pH in the range indicated for topical applications.

Buffered formulations of metronidazole suitable for vaginal introduction in accord with the present invention and suitable for achieving the desired therapeutic action and desired physiological pH of the vagina can be in any convenient non-flowing form, such as suspensions; emulsions; clear and opaque gels; semisolid systems, including ointments, pastes, oil-in-water (o/w) creams, semisolid emulsions with solid internal phases, semisolid emulsions with fluid internal phases; vaginal suppositories; tablets (inserts); and the like.

Buffered metronidazole composition vehicles suitable for use in practicing this invention may be classified as follows: (1.) Oleaginous compositional bases or ointments that are all oil, e.g., petrolatum and mineral oil systems; (2.) Absorption compositional bases; (a.) Anhydrous oleaginous systems which absorb water; (b.) Water-in-oil (w/o) emulsion systems, e.g., aquaphor; (3.) Emulsion compositional bases of the water-in-oil (w/o) type; (4.) Emulsion compositional bases of the oil-in-water type (o/w); (5.) Anhydrous water soluble compositional bases; and (6.) Suppositories/inserts.

Each of the above indicated drug delivery vehicles is known in the art; however, for exemplary purposes of preparing compositions for use in the practice of this invention, the following detailed descriptions are provided:

Oleaginous Bases or Ointments

This class of formulations include metronidazole and hydrocarbon-based semisolids containing dissolved and/or suspended bacteriostats/preservatives and a system. The petrolatum component in these bases can be any paraffin ranging in viscosity from mineral oil employing incorporated isobutylene, colloidal silica, or stearate salts to paraffin waxes. White and yellow petrolatum are examples of such systems. Bases of this class can be made by incorporating high-melting waxes into a fluid mineral oil via fusion or by incorporation of polyethylene into mineral oil at elevated temperature. Polysiloxanes (also known as silicones) are suitable for use in these bases and typically have a viscosity in the range of about 0.5 to 10⁶ centistokes. The organic entities attached to the polysiloxane are preferably lower molecular weight hydrocarbon moieties having from 1 to 8 carbons each, such as lower alkyl, lower alkenyl, phenyl and alkyl substituted phenyl, and phenyl(lower)alkyl, such as benzyl. In such a moiety, each lower alkyl or alkenyl group preferably has 1 to 3 carbons inclusive, such as in a dimethylsiloxane polymer. A specific formulation for an oleaginous system is illustrated in the examples below.

Absorption Bases

Absorption bases used for these formulations can be oleaginous systems which contain, in addition to metronidazole, ingredients with the capacity to emulsify a significant quantity of water. Water-in-oil (w/o) emulsions can be formed wherein the external phase is oleaginous in character. Preservatives/bacteriostats, such as the parabens, systems, etc. can be incorporated into these bases as emulsified aqueous solutions together with the active ingredient. Diverse additives are conveniently used as the emulsifier, and these include, but are not limited to, cholesterol, lanolin (which contains cholesterol and cholesterol esters and other emulsifiers), lanolin derivatives, beeswax, fatty alcohols, wool wax alcohols, low HLB (hydrophobe/lipophobe balance) emulsifiers, and assorted ionic and nonionic surfactants, singularly or in combination.

Water-In-Oil (W/O) Emulsion Bases

These formulations can be an expansion of the general class of absorption bases which are liquids or creams. They can be prepared by taking a mixture of metronidazole with oil phase ingredients, bacteriostats/preservatives and buffer salts which are dissolved or suspended therein and to which water has been added to form a water-in-oil emulsion.

Compositions shown in the examples below are provided as being exemplary of these systems, but those skilled in the art will appreciate that substitutions, additions, and/or omissions of the specified components can be made. A listing of alternate components that could be incorporated in these examples is provided herein below.

Oil-In-Water (O/W) Emulsion Bases

These systems are semisolid emulsions, microemulsions, or foam emulsion systems containing metronidazole. Usually such a system has a “creamy white” appearance. Typically, the internal oil phase is in the range in percentage composition of about 10% to about 40% oil by weight and the external phase may contain 80% or more water. The oleaginous phase may contain, but is not limited to, long-chain alcohols (cetyl, stearyl), long-chain esters (myristates, palmitates, stearates), long-chain acids (palmitic, stearic), vegetable and animal oils and assorted waxes. These can be made with anionic, cationic, nonionic or amphoteric surfactants, or with combinations especially of the nonionic surfactants. The examples below are exemplary of these systems, but those skilled in the art will appreciate that substitutions and additions or omissions of the specified components could be made by one who is skilled in the art. A listing of alternate components is provided below.

Anhydrous Water Soluble Bases

These systems include solutions or suspensions of metronidazole and the desired buffer system in glycols, such as glycerin, polyethylene glycol, propylene glycol which are thickened with hydroxypropyl cellulose.

The examples below are provided as being illustrative of these systems. Those skilled in the art will appreciate that substitutions, additions and/or omissions of the specified components can be made. A listing of alternate components that could be incorporated in these composition examples is provided below.

Vaginal Inserts and Suppositories

Suppositories containing metronidazole can be, for example, oleaginous in nature which melt at body temperature, or polyethylene glycol-based which dissolve in the vaginal fluids. Additional bases for suppositories are glycerin and glycerinated gelatin.

Metronidazole can be readily formulated into gels made with gelling agents. Some examples of these gelling agents include: cationic polymers, such as polyquaternium-10, which is a polymeric quaternary ammonium salt of hydroxyethyl cellulose reacted with a trimethyl ammonium-substituted epoxide, acrylate copolymers, alkyl celluloses, carboxyalkyl celluloses, carboxymethyl cellulose salts, guar gums, xanthan gum, hydroxyalkyl celluloses, poloxamers, polyvinyl alcohol, methyl vinyl ether/maleic anhydride (PVM/MA) copolymers, PVM/MA decadiene crosspolymers, carbomers (carboxyvinyl polymers), carbomer salts, acrylates/C10-30 alkyl acrylate crosspolymers, and hyaluronic acid. Preferred are carbomers and acrylates/C10-30 alkyl acrylate crosspolymers, including those commercially available from Noveon, Inc., of Cleveland, Ohio, under the designations Carbopol® and Pemulen®.

A listing below exemplifies alternate components that could be incorporated in these examples:

Surfactants

As above indicated, the formulations of this invention can contain one or more surfactants. Suitable surfactants include, e.g., anionic, cationic, amphoteric and nonionic surfactants which are pharmaceutically acceptable in topical applications. Any one or more surfactants having the above characteristics can be used. Representative examples of suitable surfactants which can be used in the formulations of this invention are described in Martin and Cook, Remington's Practice of Pharmacy, 12th edition, 1961, pp. 219-226, R. G. Harry, Cosmetics: Their Principles and Practices, (1965), pp. 396-398 and 413-417, and E. Sagarin, Cosmetics Science and Technology, (1957), pp. 328-333, 1060-1063 and 1254, which publications are herein incorporated by reference. Representative surfactants which are suitable include:

A. Anionic Agents

1. Sodium, potassium and ammonium soaps derived from fatty acids having from 10 to 22 carbon atoms; and polyvalent metal (magnesium, calcium, zinc, aluminum and lead) soaps derived from fatty acids having from 10 to 22 carbons.

2. Amine soaps derived from fatty acids having from 10 to 22 carbons and primary, secondary and tertiary amines, such as monoethanolamine, diethanolamine and triethanolamine, and cyclic amines, such as morpholine. An examples is triethanolamine stearate, or the like.

3. Rosin soaps, such as sodium salts of rosin acids, e.g., abietic acid.

4. Alkali metal salts of sulfate compounds which can be represented by the formula ROSO₃H wherein the R group represents an organic moiety, such as, for example, a fatty alcohol residue having up to 22 carbons. Examples include sodium lauryl sulfate, sodium cetyl sulfate, sodium monolauryl glyceryl sulfate, an oil such as sulfated castor, olive, teaseed, neat's foot cottonseed, rape seed, corn and rice, oil, and the like.

5. Alkali metal salts of sulfonated compounds which can be represented by the formula RSO₃H wherein the R group can have from 8 to 22 carbons. These include alkane sulfonates, such as dioctyl sodium sulfosuccinate, oxyethylated alkylaryl sulfate, alkyl aromatic sulfonates such as sodium isopropylnaphthalenesulfonate, sodium dodecylbenzenesulfonate, sodium sulfonaphthylstearate, and the like.

B. Cationic Agents

1. Amine salts (e.g., hydrochlorides and acetates) derived from straight chain fatty amines having from 8 to 18 carbons. An example is octodecylamine hydrochloride, and the like.

2. Quaternary ammonium salts formed by alkylation of fatty amines with methyl chloride, dimethylsulfate, benzylchloride, and the like. These compounds can be represented by the formula >RR′R″R′″NY wherein each of R, R′, R″, R′″ is a long chain aliphatic group of from 8 to 22 carbons or a fatty acid amide residue; a short aliphatic group, such as methyl, ethyl, or propyl, an aromatic group, such as a phenyl or benzyl radical; or a heterocyclic group, such as pyridine or piperidine residue; and Y represents an inorganic or lower organic cation, such as chloride, bromide or acetate radical. Examples include triethanolamine stearate, cetyl trimethyl ammonium bromide, benzalkoniumchloride, and the like.

C. Nonionic Agents

1. Ethers, such as condensation products of alkylphenols with from 6 to 20 moles of ethylene oxide, such phenols being monoalkylated, dialkylated or polyalkylated with alkyl side chains having from 5 to 18 carbons each, and the corresponding naphthalene or diphenyl compounds. Examples include polyoxyethylene, polyoxyethylene-polyoxypropylene copolymers, and the like.

2. Esters, such as compounds which can be represented by the formula RCOOR′ wherein R is a long hydrocarbon chain derived from a fatty acid having from 12 to 22 carbons, and R′ is derived from a polyhydric alcohol. Examples include glyceryl monostearate, diethylene glycol monolaurate, sorbitan fatty acid esters derived, for example, from lauric, palmitic, stearic and/or oleic acids, and the like.

3. Ether-esters wherein polyoxyethylene chains are found with an unreacted hydroxy group of esters of fatty acids and polyhydric alcohols.

4. Fatty acid amides, such as lauroyl diethanolamide and the like.

D. Ampholytic Agents

1. Surfactants, such as those having amino and carboxy groups. Examples include dodecyl Balanine, imidazoline derivatives such as the so-called “Miranols”, and the like.

2. Surfactants containing amino and sulfuric acid or sulfonic acid groups formed by condensing an alkanesulfonamide with formaldehyde and methyltaurine.

Suitable representative surfactants from the above indicated four general classes include sorbitan trioleate, sorbitan tristearate, sorbitan sesquioleate, glycerol monostearate, sorbitan monostearate, sorbitan monopalmitate, sorbitan monolaurate, polyoxyethylene lauryl ether, polyethylene glycol 400 monostearate, triethanolamine oleate, polyoxyethylene glycol 400 monolaurate, polyoxyethylene sorbitan monostearate, polyoxyethylenesorbitan monooleate, polyoxyethylene sorbitan monolaurate, sodium oleate, potassium oleate, sodium lauryl sulfate, lauroyl imidazoline, sodium dodecylbenzene sulfonate, sodium monoglyceride sulfate, sodium alkaralkyl polyglycol sulfate, sodium oleyl taurate, sodium dioctyl sulfosuccinate, lauryl polyglycol, ether, sodium dibutylnaphthalenesulfonate, alkyl phenol polyglycol ether, sorbitan monolaurate polyglycol ether, sulfonated castor oil, tall oil polyglycol ester, alkyl dimethyl benzylammonium chloride, alkyl naphthalene pyridinium chloride, cetyl dimethyl ethylammonium bromide, alkyl dimethyl chlorobenzylammonium chloride, dibutyl phenyl phenol sulfonate, ester of colaminoethylformyl methylpyridinium chloride, sulfonated methyl oleylamide, sorbitan monolaurate polyglycol ether, polyglycol oleate, sodium lauryl sulfoacetate, sodium 2-ethylhexanol sulfate, sodium 7-ethyl-2-methylundecanol-4 sulfate, sodium 3,9-diethyltridecanol-6 sulfate, sodium lauryl and myristyl collamide sulfonate and N-(sodium sulfoethyl) oleamide, and the like.

Preservatives

As above indicated, the compositions of this invention can include suitable bacterostats, preservatives, inhibitors, or the like, such as methyl, ethyl, propyl, and butyl esters of parahydroxybenzoic acid, propyl gallate, sorbic acid and its sodium and potassium salts, propionic acid and its calcium and sodium salts, “Dioxin” (6-acetoxy-2,4-dimethyl-m-dioxane), “Bronopol” (2-bromo-2-nitropropane-1,3-diol) and salicylanilides such as disbromosalicylanilide, tribromosalicylamilides, “Cinaryl” 100 and 200 or “Dowicil” 100 and 200 (Cis isomer of 1-(3-chloroallyl-3,5,7-triaza-1-azanidadamantane chloride), hexachlorophene, sodium benzoate, citric acid, ethylene diaminetetraacetic acid and its alkali metal and alkaline earth metal salts, butyl hydroxyanisol, butyl hydroxytoluene, phenolic compounds such as chloro- and bromocresols and chloro- and bromo-oxylenols, quaternary ammonium compounds like benzalkonium chloride, aromatic alcohols such as phenylethyl alcohol, benzyl alcohol, etc., chlorobutanol, quinoline derivatives such as iodochlorhydroxyquinolin, and the like.

Hydrophilic and Hydrophobic Thickeners (Suspending Gelling or Viscosity Inducing Agents)

Suitable thickeners which may be used in the composition of this invention include colloidal alumina, colloidal silica, alginic acid and derivatives thereof, “Carbopols” (carboxyvinyl polymers), cellulose derivatives, such as “Klucel” (cellulose ethers), Methocel (methyl cellulose), “Natrosol” (hydroxyethyl cellulose), sodium carboxymethyl cellulose, gelatin, natural gums, such as agar, tragacanth, acacia gum, guar gum, stearates, isobutylene, waxes, carrageen, and the like, egg yolk, lecithin, pectin, thixcin, resins like ethyleneoxide polymers, such as the so called polyoxes, and the like.

Other Adjuvants/Cosolvents

Other adjuvants which can be incorporated into a composition of this invention includes waxes, such as beeswax, spermaceti, paraffin waxes, and fatty acids, alcohols and amides having from 10 to 22 carbons, and the like.

Monohydric alcohols can be used, such as those having from 1 to 22 carbons per molecule, such as methanol, ethanol, propanol, isopropanol, butanol, hexanol, cetyl alcohol, stearyl alcohol, and the like.

Dihydric and polyhydric alcohols can be used, such as those having from 2 to 22 carbons per molecule, such as propylene glycol, glycerin, hexanetriols, such as 1,2,6-hexanetriol, sorbitol, 1,3-butanediol, 2,3-butanediol, and the like.

Polyethylene glycols and polypropylene glycols can be used, such as those having molecular weight in the range of about 100 to about 20,000.

Esters of aliphatic monobasic and dibasic acids can be used, such as those having from 2 to 22 carbons per molecule, with (a) monohydric alcohols having from 1 to 20 carbons per molecule, (b) di- and polyhydric alcohols having from 2 to 20 carbons per molecule, and (c) sugar alcohols. Examples include isopropyl myristate, myristyl myristate, cetyl stearate, methyl stearate, isopropyl sebacate, methyl sebacate, sucrose monolaurate, sucrose monostearate, and the like.

Buffers

In general, and as above indicated, buffers for the present compositions can include any physiologically acceptable base (e.g., inorganic base, organic base, or combination thereof). The base can include, but is not limited to, sodium hydroxide, potassium hydroxide, lithium hydroxide, calcium hydroxide, sodium carbonate, sodium bicarbonate, potassium carbonate, potassium bicarbonate, and the like.

Gases

Compositions of this invention can contain air or some other medically/pharmaceutically/cosmetically acceptable gas which is emulsified in a liquid phase of such composition to provide a foam.

Illustrative Compositions of Metronidazole

A composition of the invention advantageously includes, in general, at least about 0.1 weight percent metronidazole, based on the total weight of the composition. Preferably metronidazole is present in an amount in the range of about 0.1% to about 2%, more preferably in an amount in the range of about 0.25% to about 1%, and most preferably about 0.75% by weight, based on the total weight of the composition. Larger and smaller contents of metronidazole can be used without departing from the spirit and scope of this invention, however.

Substantially oil-free, aqueous compositions containing metronidazole, in which this drug is solubilized in a single-phase aqueous gel, are a preferred class of embodiments used in the practice of this invention. The overall advantages of such aqueous gel compositions in treating BV have been discussed above, and are presented and illustrated in greater detail herein below.

The actual concentration of metronidazole in any given such composition may vary, depending on variables such as the nature and degree of the BV being treated, the duration of the therapeutic treatment period contemplated, the size of the particular unit dose to be administered, and the like.

In the preferred compositions, metronidazole is in an aqueous solution of a high molecular weight polycarboxylated vinyl polymer. The polymer imparts a desirable viscous, gelled consistency to the composition when mixed with metronidazole and water. The preferred gel compositions contain at least about 95% by weight water, based on the total weight of the composition, and have the requisite degree of metronidazole concentration, and hence thermodynamic activity, for effective topical delivery and bioavailability of metronidazole in the vagina. The preferred gel compositions also have the requisite therapeutic activities as previously described.

The gel-forming polymer useful in compounding such preferred compositions may be any suitable polymer which is hydrophilic and water-dispersible, has free carboxylic groups and relatively high base binding capacity, and forms an aqueous gel of substantially uniform consistency when neutralized with a base. Preferred polymers for use in the compositions of the invention are water-dispersible, polycarboxylated vinyl polymers. Polyacrylic acid polymers are particularly preferred for the present purposes. The molecular weight of the polymer is desirably in the range of about 1,250,000 and about 4,000,000 daltons. Suitable polyacrylic acid polymers include, but are not limited to, polyacrylic acid polymers slightly cross-linked with a polyalkenyl polyether, such as those commercially available from Noveon, Inc., Cleveland, Ohio, under the trademarks Carbopol 934, 934P, 940, 941, 974, 974P, 980, 981, 1342, and 1382. Carbopol 934P® is a particularly preferred polymer for use in practicing this invention.

The polymer is present in an amount sufficient to cause gelling of a preferred composition, and to impart the desired viscous consistency to the resulting topical formulation. In addition and importantly, the polymer is used in concentrations that afford the buffering capacity and pH range that are necessary for this method. The metronidazole compositions advantageously include about 0.2% to about 7% by weight of the polymer, preferably about 0.5% to about 2.5%, and most preferably about 2% by weight of the polymer based on the total weight of the composition.

Aqueous solutions of these polymers form gels when neutralized with a base. Water-soluble bases which have been used to promote gelling of such polymers as the Carbopols® include, for example, inorganic bases, such as an aqueous solution of ammonia, NaOH, and organic amine, e.g., alkylamines, such as methylamine and ethylamine, dialkylamines, trialkylamines, alkanolamines, dialkanolamines, and the like. Preferably a strong base is employed. The pharmaceutically effective component of the compositions of the present invention, metronidazole, is itself sufficiently basic to partially neutralize the acidic polymer in aqueous solution to the desired degree and to promote gelling.

A preferred gel composition can further optionally include a solubilizer, i.e., an agent that promotes penetration of the active drug into the microorganisms. Such solubilizers include: benzyl alcohol, benzyl benzoate, butoxydiglycol, diacetin, triacetin, propylene glycol, polyethylene glycol, propylene glycol butyl ether, glycerin, hexylene glycol, dipropylene glycol, ethoxydiglycol, ethoxydiglycol acetate, dimethyl isosorbide, dimethyl sulfoxide, and propylene carbonate. Propylene glycol is preferred. The composition advantageously includes about 1% to about 50%, preferably about 2% to about 5%, and more preferably about 3% by weight, of such solubilizer, based on the total weight of the composition.

Preservatives optionally can be incorporated into such gel compositions in an amount effective for inhibiting growth of microbes, such as yeast, molds, and bacteria during gel composition storage. Any conventional preservative can be used, with parabens being preferred. A mixture of methyl paraben and propyl paraben has been found to be particularly effective as a preservative. Most preferably, such a composition comprises about 0.08% by weight of methyl paraben and about 0.02% by weight of propyl paraben based on the total weight of the gel composition.

Ethylenediaminetetraacetic acid (EDTA) or one of its salts is commonly added to dermatological preparations, and may optionally be incorporated into the gel composition. EDTA chelates certain metals that may be present in the formulation, which is useful because some patients have adverse reactions to preparations containing metal impurities. The EDTA will also inhibit undesirable “browning” of the composition which may occur over time in compositions having a low pH value, e.g., a pH value of about 3 to about 4.5. Advantageously, a gel composition optionally further includes from about 0.01% to about 0.1%, preferably about 0.05% by weight, of EDTA based on the total weight of the composition.

The final pH value of a gel composition may vary within the physiologically compatible range. Advantageously, the final pH value is a physiologically compatible, i.e., not harmful to biological tissue, adjusts and controls vaginal environment to normal, healthy range and is acidic. The preferred pH value is about 5.0 to about 6.0, more preferably about 5.5. Any suitable method of adjusting the pH value of aqueous solutions may be used. Advantageously, sodium hydroxide (NaOH) is added to the composition to bring the final pH value to the desired level. The gel compositions are more viscous at pH values that approach neutrality than at the more acidic pH values within the preferred range, i.e., viscosity increases as the polymer in the gel is neutralized to a greater degree, e.g., with NaOH.

The ingredients listed above may be combined in any order and manner that produces a composition comprising metronidazole dissolved in, and evenly dispersed throughout, a one-phase aqueous gel of the desired consistency and pH value. One suitable method of preparing such compositions involves preparation of an aqueous solution of the polymer, which will be called “Part A”. Advantageously, this solution includes the polymer in distilled water. A “Part B” is prepared comprising metronidazole. Mixing of Parts A and B results in gelling of the composition. The optional solubilizer and preservative(s) are preferably included in Part B. If EDTA is to be added to the formulation, it is preferably included in Part A. The pH value may then be adjusted to the desired level, e.g., by addition of NaOH.

The resulting homogeneous gels having a pH in the range indicated possess the advantageous properties described above, including utilizing noninflammatory and non-irritating ingredients. Higher specific activity of metronidazole results due to increased diffusion across membranes, release from the vehicle, and controlled pH. The result is greater therapeutic effectiveness using smaller amount of metronidazole. A formulation has a desirable consistency that prevents undesirable pooling and leaking of metronidazole. High concentrations of tissue-drying ingredients (e.g. alcohols and acetone), which are found, for example, in some preparations to promote drug solubility, are also avoided. Such ingredients at high concentration may excessively dry the patient's vaginal wall causing undesirable discomfort.

As indicated above, when such above described gel composition is introduced as described into an afflicted vagina, a prolonged and surprisingly uniform and regulated (controlled) release rate of metronidazole from the gel composition into the environment of the vagina is achieved. Pooling and running is minimized. The release rate or delivery is sustained for an extended period of time.

The release rate is such that the quantity of the drug which is delivered to vaginal tissues during the release period is at, or slightly above, a minimum therapeutically effective level.

The gel composition also has an unusual and very useful buffering capacity which, in addition to, and in coaction with, the desired bactericidal activity of the metronidazole, is desirable and important in achieving the therapeutic effectiveness that is associated with the practice of this invention. This combination allows for the therapeutic effectiveness of the novel low dose metronidazole formulation by adjusting and controlling the pH of the vaginal environment.

Thus, the gel compositions, as is characteristic of a composition of the invention generally, resist changes in pH upon exposure in the use environment to an acid or a base. In the preparation of a gel composition as above explained herein, a strong base (e.g., sodium hydroxide) is preferably added to the Carbopol® polymer (weak acid form). This neutralization thickens the formulation to produce the desired gel consistency. It also produces the mixture of components needed to produce a buffered system.

As the exemplary material herein below presented indicates, when a portion of a gel formulation is titrated by a strong base (e.g., sodium hydroxide) successively using each of a concentrated solution of the base and a dilute solution of the base, such that the total volume of base is substantially increased (for example, doubled), it is found not only that there is a significant buffering effect inherent in the gel formulation, but also that there is very little effect on the gel formulation buffer strength as a result of dilution.

These results are significant for purposes of accomplishing topical treatment of, for example, BV by the practice of this invention. For one thing, these results show that the inherent dilution of a unit dose of gel composition which occurs in the vagina does not affect the ability of the gel composition to help prevent and to treat the undesirable alkalinization of the vaginal tissue caused by infections of the BV type. For another thing, these results show that vaginal tissue can be promoted to remain at a pH below about 4.5 which is desirable to inhibit BV organism activity, and to promote certain desirable and normal bacterial colonization and development, such as hydrogen peroxide producing Lactobacilli (Lactobacillus H₂O₂+), and the like.

The practice of the present invention is demonstrated in the following examples. These examples are meant to illustrate the invention rather than to limit its scope. Variations in the treating compositions which do not adversely affect the effectiveness of metronidazole will be evident to one skilled in the art, and are within the scope of this invention. For example, additional ingredients such as coloring agents, and the like may be included in the compositions as long as the resulting composition retains desirable properties, as described above. Unless otherwise indicated, each composition is prepared by conventionally admixing the respective indicated components together. Also, unless otherwise indicated, each composition is prepared using a buffer (buffer system) which in use provides a pH value in the range of about 5.0 to about 6.0.

### EXAMPLES

### Example 1

### A Multi-Center, Randomized, Double-Blind, Parallel Group Study Comparing the Bioequivalence of Atrix Laboratories, Inc. Generic Formulation of Metronidazole Vaginal Gel, 0.75% and MetroGel-Vaginal® Metronidazole Vaginal Gel, 0.75% in the Treatment of Bacterial Vaginosis

The study compared the efficacy, safety, and tolerance of Atrix Laboratories, Inc. generic formulation of metronidazole vaginal gel, 0.75% and 3M Pharmaceuticals' MetroGel-Vaginal® metronidazole vaginal gel, 0.75% in the treatment of bacterial vaginosis.

Introduction

Bacterial vaginosis (BV) is the most common cause of vaginitis in women of childbearing age, causing 40-50% of all vaginal infections. Subjects present with an unpleasant, “fishy smelling” off-white, thin, and homogenous discharge without an apparent inflammatory response. The disease represents a complex change in the vaginal flora with a reduction in the prevalence and concentration of lactobacilli (especially hydrogen peroxide producing forms), and a concomitant increase in Gardnerella vaginalis, Mobiluncus spp., anaerobic Gram-negative rods (of the genera Bacteroides, Prevotella, and Porphyromonas), Peptostreptococcus spp. and Mycoplasma hominis. Bacterial vaginosis is implicated in recurrent urinary tract infections, pre-term labor, and a variety of upper genital tract infections including postpartum endometritis, post-hysterectomy and post-abortion infection, and pelvic inflammatory disease.

Predisposing factors associated with bacterial vaginosis are non-white ethnicity, prior pregnancy, use of an IUD, sexual activity, new sexual partners, and recent antibiotic use. It is also associated with concurrent trichomoniasis and/or the absence of hydrogen peroxide producing lactobacilli.

Materials and Methods

The study period was 22-29 days and the treatment period for each subject was 5 days. The test product was Metronidazole vaginal gel, 0.75%, Atrix Laboratories, Inc. and the comparative therapy was MetroGel-Vaginal® metronidazole vaginal gel, 0.75%, 3M Pharmaceuticals.

Both the test product and comparative therapy were supplied in tubes that contained 70 grams of vaginal gel. Each gram of active gel contained 7.5 mg of metronidazole. Subjects administered one applicator full of vaginal gel (approximately 37.5 mg of metronidazole) with each dose using the supplied 5-gram vaginal applicators.

Metronidazole vaginal gel, 0.75%, Atrix Laboratories, Inc. was prepared using the specific weights of reagents illustrated in Table 1.

TABLE 1
Test Product Composition

| Component | Tradename | % w/w |
|---|---|---|
| Metronidazole, USP | Metronidazole | 0.75 |
| Propylene Glycol, USP | Propylene Glycol | 3.0 |
| Methylparaben, NF | Methylparaben | 0.08 |
| Propylparaben, NF | Propylparaben | 0.02 |
| Edetate Disodium, USP | Edetate Disodium | 0.05 |
| Sodium Hydroxide, NF | Sodium Hydroxide | 0.25 |
| Carbomer 934P, NF | Carbopol 934P | 2.0 |
| Purified Water, USP | Purified Water | 93.85 |

The resulting pH of the composition is typically 5.50 (all pH measurements recorded dilute in purified water at 10:1 dilution).

Study Population:

This multi-center study was comprised of subjects presenting with a clinical diagnosis of bacterial vaginosis that was suitable for treatment with an intra-vaginal antibiotic. Female subjects 18 years of age or older, of any race, who met the inclusion criteria (a confirmed clinical diagnosis of bacterial vaginosis) were enrolled. Subjects who, after the pelvic exam, met the inclusion/exclusion criteria were randomly assigned in a 1:1 ratio to one of the two study formulations. The study included 382 per-protocol subjects. One hundred ninety-eight subjects received Atrix Laboratories, Inc.'s metronidazole vaginal gel, 0.75% and 184 subjects received 3M Pharmaceuticals' MetroGel-Vaginal® metronidazole vaginal gel, 0.75%. A statistician not directly involved with the study performed the generation of the randomization schedule.

Three subject populations were defined:
1) An intent-to-treat (ITT) subject was any subject who received study medication and returned for at least one follow-up visit.
2) A modified intent-to-treat (mITT) subject was any subject who received study medication, returned for at least one follow-up visit, had a negative test for Neisseria gonorrhoeae, Chlamydia trachomatis, and a Gram's stain slide Nugent Score ≧4 at Visit 1.
3) A per-protocol (PP) subject was any subject who met inclusion and exclusion criteria, began therapy within 48 hours of Visit 1, was compliant with study medication (received at least 3 consecutive days of therapy and no more than 6 days of therapy), had no study violations which could have altered the effect of, or the accurate assessment of, the applied study treatment, and was assessed for efficacy at Visit 3.

If the Baseline Visit (Day 1) LCx assay results were positive for Neisseria gonorrhoeae or Chlamydia trachomatis, or the Baseline Visit Nugent Score was 0-3, the subject was discounted from the study. No subject with known or suspected other infectious causes of vulvovaginitis (e.g. candidiasis, Trichonomas vaginalis, active Herpes simplex, or human papilloma virus) or other conditions that would confound the interpretation of clinical response were included in the study.

Design:

Subjects in a double-blind parallel group study were randomly assigned to either Atrix Laboratories, Inc.'s generic formulation of metronidazole vaginal gel, 0.75% or 3M Pharmaceuticals' MetroGel-Vaginal®. Clinical evaluations were preformed at: Baseline Visit (Day 1); Post-Treatment Telephone Contact (Visit 2), which occurred 7 to 10 days after the first day of treatment (Day 8 to Day 11); and Test-of-Cure Visit (Visit 3), which occurred 21 to 28 days after the first day of treatment (Day 22 to Day 29). At Visit 3, subjects were examined and classified as a Clinical Cure or Clinical Failure, and Bacteriological Cure or Bacteriological Failure.

Subjects began therapy within 48 hours of the Baseline Visit (Day 1). The medication was to be applied vaginally once daily at bedtime for five consecutive days using the supplied 5-gram vaginal applicators. Subjects received at least three consecutive days of therapy, but not more than 6 total days of therapy, to have been considered per-protocol.

Baseline Visit (Day 1)

At the Baseline visit (Day 1), once a presumptive diagnosis of bacterial vaginosis was made, the investigator performed a medical history and pelvic exam. A PAP Smear was performed if no clinical results from previous 12 months were available. Specimens were collected for each of the following tests:
Test 1—saline “wet mount” to check for the presence of clue cells and Trichonomas vaginalis,
Test 2—10% KOH “whiff test”,
Test 3—Vaginal fluid pH
Test 4—Gram's stain (the slide was sent to a central reference lab for Nugent scoring),
Test 5—Urine pregnancy,
Test 6—Chlamydia trachomatis by LCx assay, and
Test 7—Neisseria gonorrhoeae by LCx assay.

The saline wet mount (Test 1) was examined for the presence of clue cells and Trichomonas vaginalis. Clue cells must have been ≧20% of the total epithelial cells on microscopic examination for the subject to participate in the study. If T. vaginalis was identified on the wet mount, the subject was excluded from participating in the study.

The whiff test (Test 2) was performed using the 10% KOH solution “whiff test”. Subjects must have had a positive pH test (pH>4.5) and a positive “whiff test” (a fishy odor of the vaginal discharge with the addition of a drop of 10% KOH solution) to be included in the study.

The pH test (Test 3) was performed using ColorpHast pH paper. The slide collected from the Gram's stain (Test 4) was assigned a Nugent Score according to Table 2.

TABLE 2
Nugent Scoring System For Gram's Stained Vaginal Smears

| | Lactobacillus | Gardnerella/Bacteroides | Curved Gram- |
|---|---|---|---|
| SCORE* | morphotypes | spp. Morphotypes | variable rods |
| 0 | **4+ | 0 | 0 |
| 1 | 3+ | 1+ | 1+ or 2+ |
| 2 | 2+ | 2+ | 3+ or 4+ |
| 3 | 1+ | 3+ | |
| 4 | 0 | 4+ | |
| *Morphotypes were scored as the average number seen per oil immersion field (minimum of 10-20 fields were examined). Each morphotype was then given a score from the left hand column. The TOTAL SCORE was calculated by adding the individual morphotype | | | |
| # scores = Lactobacillus + Gardnerella/Bacteroides + Curved Gram-negative rods. | | | |

**QUANTIFICATION SCALE: 0 = no morphotypes seen; 1+ = <1 morphotype per field; 2+ = 1 to 4 morphotypes; 3+ = 5 to 30 morphotypes; 4+ = >30 morphotypes per field.

The LCx GC/Chlamydia detection system was used to test for the presence of Neisseria gonorrhoeae and Chlamydia trachomatis (Tests 6 and 7). The LCx GC/Chlamydia detection system used one swab for detection of both pathogens.

The following written Instructions and Precautions were given to each subject at the Baseline Visit:

### Subject Instructions

1. Begin the study medication on the day of your first study visit, unless otherwise directed by study personnel.
2. To prepare the medication for application, first remove the cap from the tube and puncture the tamperproof seal with the sharp end of the tube cap. Screw on one of the supplied plastic applicators with the plunger in the down position. Fill the applicator by squeezing the tube until the applicator is full. Unscrew the applicator from the tube.
3. Insert the applicator into the vagina and depress the applicator plunger to apply the medication. This may be most easily done while lying on your back. The applicator should then be discarded.
4. You will use the medication once daily at bedtime for 5 days (5 doses).
5. Record all doses taken on the diary card provided.
6. Do not expose the study medication to extremes in temperature and do not attempt to remove the black shrink-wrap from the medication tube.
7. Please discard all applicators, used and unused, and return the study medication at your next study visit.

### Precautions

Metronidazole vaginal gel contains ingredients that may cause burning and irritation of the eye; therefore, contact with the eyes should be avoided. In the event of accidental contact with the eye, rinse the eye with copious amounts of cool tap water.

You should not drink alcohol during the five-day treatment period and for one day afterward. Alcohol taken with oral metronidazole can cause nausea and vomiting. While blood levels are significantly lower with metronidazole vaginal gel than with usual doses of oral metronidazole, a possible interaction with alcohol cannot be excluded.

You should not engage in vaginal intercourse throughout the first 7 days of the study.

Test-Of-Cure (TOC) Visit (Day 22-29)

Subjects returned to the study center for a Test-of-Cure Visit (Visit 3), 21 to 28 days after the first day of treatment. A gynecological exam was performed. Laboratory testing at the TOC Visit consisted of Tests 1-5, described above. The clinical response and bacteriological response of each subject was assessed.

The primary efficacy endpoint was the therapeutic cure rate, which included both the clinical response and the bacteriological response (Nugent Score), of each subject at the TOC Visit. The secondary efficacy endpoints were the therapeutic cure rate for the modified intent-to-treat (mITT) subjects, clinical cure proportions for the per-protocol (PP) and modified intent-to-treat (mITT) subjects, and bacteriological cure proportions for the per-protocol (PP) and modified intent-to-treat (mITT) subjects.

A subject who was assessed as both a clinical cure and bacteriological cure (Nugent score of 0-3 at the TOC visit) was considered a therapeutic cure. A subject assessed as either a clinical failure or bacteriological failure was considered a therapeutic failure. The subjects were classified as a Clinical Cure or Clinical Failure, and Bacteriological Cure or Bacteriological Failure using the following definitions:

A. Clinical Response

Clinical Cure: Clinical cure was defined as resolution of the clinical findings from the Baseline Visit. To fall under the classification of a Clinical Cure, subjects must have had all of the following: an original discharge characteristic of bacterial vaginosis that had returned to a normal physiological discharge, which varied in appearance and consistency depending on the menstrual cycle; a negative Test 1; a negative Test 2; and a Test 3 result of <4.7 (vaginal fluid pH of <4.7).

Clinical Failure: Clinical failure was defined as a subject who did not meet the definition of clinical cure.

B. Bacteriological Response (Test 4—Nugent Score)

Bacteriological Cure: Bacteriological Cure was defined as a Nugent Score <4.

Bacteriological Failure: Bacteriological Failure was defined as a Nugent Score >4.

Statistical Methods:

A. Sample Size Rationale and Significance Level:

Atrix Laboratories, Inc.'s generic formulation of metronidazole vaginal gel 0.75% was evaluated to determine if it was bioequivalent to MetroGel-Vaginal® based on a two one-sided test evaluation of the proportions of subjects with therapeutic cure at Visit 3 (the so-called “Test of Cure” visit). The procedure was evaluated at the 5% level of significance (α=0.05). Sample size was based on information obtained from the MetroGel-Vaginal® once-per day (QD) formulation.

The Summary Basis of Approval test procedure was constructed as a two-sided 90% Wald's confidence interval (α=0.05 in each tail), with Yate's continuity correction to have approximately 90% power, on the difference between treatment therapeutic cure proportions. For an asymptotically normal 90%, continuity-corrected confidence interval about the difference in success proportions between the Atrix Laboratories, Inc. (test) and Metro-Gel-Vaginal® (reference) products, covering a maximum allowable difference of 0.20, a minimum of 163 per-protocol subjects per treatment group was required. This was based on an expected therapeutic cure rate of 53%. The calculation allows for the possibility of the true cure rate difference between products ranging from −3% to +3% of the reference product cure rate.

The calculation of Wald's 90% confidence interval with Yate's continuity correction included only the results of the per-protocol subjects. If the 90% confidence interval for the difference in cure proportions was contained within ±0.20 (±20%) then the test product was judged bioequivalent to the reference product. The Intent-to-Treat cohort was analyzed in similar fashion to determine the consistency of the per-protocol subject findings. A last-observation-carried-forward approach was used for missing Intent-to-Treat efficacy measures.

B. Secondary Statistical Analyses:

Confidence intervals for the difference between the test and reference treatment groups for the following secondary evaluations of efficacy were reported (see Tables 2-4):
Therapeutic cure rate for the modified intent-to-treat (mITT) subjects;
Clinical cure proportions for the per-protocol (PP) and modified intent-to-treat (mITT) subjects; and
Bacteriological cure proportions for the per-protocol (PP) and modified intent-to-treat (mITT) subjects.

C. Formula for Evaluating Bioequivalence of Test and Reference Products:

The bioequivalence analysis compared the two active treatments to show comparability between the success rates for the test and reference products.

Let Π_{R} denote the true success proportion for the reference product and Π_{T} denote the true success proportion for the test product. Let Δ=0.20 (20%) be the maximum difference worth detecting as the criterion for equivalence.

The bioequivalence analysis tested the following two one-sided hypotheses for the test and reference products:

1) H₀: Π_{T}>Π_{R}+Δ or Π_{T}−Π_{R}>Δ

vs.

H_{A}: Π_{T}≦Π_{R}+Δ or Π_{T}−Π_{R}≦Δ

2) H₀: Π_{T}<Π_{R}−Δ or Π_{T}−Π_{R}<−Δ

vs.

H_{A}: Π_{T}≧Π_{R}−Δ or Π_{T}−Π_{R}≧−Δ.

Hypotheses 1 and 2 was shown to be met if the 90% continuity-corrected confidence interval for Π_{T}−Π_{R} was contained within the interval −Δ to +Δ. Wald's 90% confidence interval incorporating Yate's continuity correction was calculated as:
(p_{T}−p_{R})±(1.645*SE+0.5*(1/n_{T}+1/n_{R})

where,
p_{T} and p_{R}=Test and Reference therapeutic cure proportions
q_{T} and q_{R}=(1−p_{T}) and (1−p_{R}), respectively
n_{T} and n_{R}=Test and Reference subject numbers, respectively
SE=Sqrt[(p_{T}q_{T}/n_{T})+(p_{R}q_{R}/n_{R})].
   Results and Discussion

The study results are illustrated by the data shown below in Tables 3-5. A therapeutic cure rate of 31.3% was found for the per-protocol test population compared to 29.9% of the reference population. The test product was judged bioequivalent to the reference product. A 90% confidence interval for the difference in cure proportions was contained within ±0.20 (±20%).

TABLE 3
Comparison of the Therapeutic Cure Rate between Treatments

| Parameter/ | | Test | | | Reference | | Difference | | |
|---|---|---|---|---|---|---|---|---|---|
| Population | Cured | Population* | (%) | Cured | Population* | (%) | (Test − Reference) | 90% CI | |
| Modified ITT | 76 | 252 | (30.2%) | 68 | 248 | (27.4%) | 2.74 | (−4.318, | 9.797) |
| Per-Protocol | 62 | 198 | (31.3%) | 55 | 184 | (29.9%) | 1.42 | (−6.862, | 9.705) |

*Includes all non-missing data, values of “N/A” are counted as treatment failures.

TABLE 4
Comparison of the Clinical Cure Rate between Treatments

| Parameter/ | | Test | | | Reference | | Difference | | |
|---|---|---|---|---|---|---|---|---|---|
| Population | Cured | Population* | (%) | Cured | Population* | (%) | (Test − Reference) | 90% CI | |
| Modified ITT | 137 | 245 | (55.9%) | 125 | 245 | (51.0%) | 4.90 | (−2.914, | 12.710) |
| Per-Protocol | 114 | 198 | (57.6%) | 94 | 184 | (51.1%) | 6.49 | (−2.409, | 15.387) |

*Includes all non-missing data, values of “N/A” are counted as treatment failures.

TABLE 5
Comparison of the Bacteriological Cure Rate between Treatments

| Parameter/ | | Test | | | Reference | | Difference | | |
|---|---|---|---|---|---|---|---|---|---|
| Population | Cured | Population* | (%) | Cured | Population* | (%) | (Test − Reference) | 90% CI | |
| Modified ITT | 82 | 245 | (33.5%) | 80 | 245 | (32.7%) | 0.82 | (−6.583, | 8.216) |
| Per-Protocol | 66 | 198 | (33.3%) | 62 | 184 | (33.7%) | −0.36 | (−8.837, | 8.113) |

*Includes all non-missing data, values of “N/A” are counted as treatment failures.

1. A buffered non-flowing composition suitable for the treatment of bacterial vaginosis which comprises metronidazole in a concentration of about 0.50% (w/w) to about 1.50% (w/w), and the metronidazole is present together with a buffer system in a physiologically tolerable medium; said buffer system providing an acidic buffered pH value for the composition in the range of about 5.0 to about 6.0.

2. The composition of claim 1 in a gel dosage form.

3. The composition of claim 1 wherein said composition comprises metronidazole in a treatment amount of about 375 mg or less.

4. The composition of claim 1 in the form of a unit dose comprising metronidazole in an amount in the range of about 20 to about 100 milligrams.

5. The composition of claim 1 in the form of a unit dose comprising metronidazole in an amount in the range of about 20 to about 40 milligrams.

6. The composition of claim 1 in the form of a unit dose comprising metronidazole in an amount of about 37.5 milligrams.

7. The composition of claim 1 which has a viscosity at least sufficient to maintain said composition in a substantially non-flowable state at ambient conditions.

8. The composition of claim 1 wherein the concentration of metronidazole is in the range of about 0.25 percent to about 1 percent by weight, based on the total weight of the composition.

9. The composition of claim 1 wherein the concentration of metronidazole is in the range of about 0.1 percent to about 2 percent by weight, based on the total weight of the composition.

10. The composition of claim 1 wherein said composition comprises about 0.75 weight percent metronidazole, based on the total weight of the composition.

11. A gel composition comprising:
(a) an antibiotic agent, an antifungal agent, or a combination thereof;
(b) a base;
(c) a gelling agent; and
(d) a solvent;
the composition having a pH of about 5.0 to about 6.0.

12. The gel composition of claim 11, wherein the antibiotic agent, an antifungal agent, or a combination thereof is water-soluble.

13. The gel composition of claim 11, wherein the antibiotic agent, an antifungal agent, or a combination thereof is active against Bacterial vaginosis (BV).

14. The gel composition of claim 11, wherein the antibiotic agent, an antifungal agent, or a combination thereof is active against Gardnerella vaginalis, Mycoplasma hominis, anaerobic bacteria including Bacteroides, Peptococcus, and Mobiluncus; Mobiluncus mulieris, Mobiluncus curtisii, G. vaqinalis, Peptococcus species, Peptococcus asaccharolvticus, Peptostrepotococcus anaerobius, Grampositive anaerobic coccus, Bacteroides species, or a combination thereof.

15. The gel composition of claim 11, wherein the antibiotic agent comprises a sulphonamide, penicillin, tetracyline, chloramphenicol, aminoglycoside, macrolide, glycopeptide, streptogramin, quinolone, fluoroquinolone, oxazolidinone, or any combination thereof.

16. The gel composition of claim 11, wherein the antibiotic agent comprises metronidazole, clindamycin, a pharmaceutically acceptable salt thereof, or a combination thereof.

17. The gel composition of claim 11, wherein the antibiotic agent comprises amoxicillin, amoxicilin sodium, ampicillin, ampicillin sodium, ciprofloxacin, ciprofloxacin hydrochloride, clarithromycin, clindamycin hydrochloride, clindamycin phosphate, erythromycin, lincomycin hydrochloride, metronidazole, or a combination thereof.

18. The gel composition of claim 11, wherein the antifungal agent comprises butenafine hydrochloride, naftifine hydrochloride, terbinafine hydrochloride, terconazole, or a combination thereof.

19. The gel composition of claim 11, wherein the antibiotic agent, an antifungal agent, or a combination thereof is present up to about 1.5% (w/w) of the composition.

20. The gel composition of claim 11, wherein the base is an organic base.

21. The gel composition of claim 11, wherein the base is an inorganic base.

22. The gel composition of claim 11, wherein the base is an inorganic base selected from the group of potassium hydroxide, sodium hydroxide, lithium hydroxide, and combinations thereof.

23. The gel composition of claim 11, wherein the base is present in an amount sufficient to adjust the pH of the composition to about 5.0 to about 6.0.

24. The gel composition of claim 11, wherein the base is present in an amount sufficient to adjust the pH of the composition to about 5.3 to about 5.7.

25. The gel composition of claim 11, wherein the gelling agent comprises a cationic polymer, an acrylate copolymer, an alkyl cellulose, a carboxyalkyl cellulose, a carboxymethyl cellulose salt, a guar gum, xanthan gum, a hydroxyalkyl cellulose, a poloxamer, polyvinyl alcohol, a methyl vinyl ether/maleic anhydride (PVM/MA) copolymer, a PVM/MA decadiene crosspolymer, a carbomer (polyacrylic acid (PPA)), a carbomer salt, a acrylates/C10-30 alkyl acrylate crosspolymer, hyaluronic acid, or any combination thereof.

26. The gel composition of claim 11, wherein the gelling agent comprises a polyacrylic acid (PAA).

27. The gel composition of claim 11, wherein the gelling agent comprises carbomer 934P.

28. The gel composition of claim 11, wherein the gelling agent is present in an amount sufficient to gel the composition.

29. The gel composition of claim 11, wherein the solvent comprises water, propylene glycol, or any combination thereof.

30. The gel composition of claim 11, further comprising a humectant.

31. The gel composition of claim 11, further comprising a humectant selected from the group of, a glycol, propylene glycol, ethylene glycol, glycerin, a polyhydric alcohol, triethylene glycol, tetraethylene glycol, sorbitol, or a combination thereof.

32. The gel composition of claim 11, further comprising a humectant present up to about 20% (w/w) of the composition.

33. The gel composition of claim 11, further comprising a preservative.

34. The gel composition of claim 11, further comprising a preservative selected from the group of, methylparaben, propylparaben, quat-15, parabens, dichlorobenzyl alcohol, ethylene diamine tetreacetic acid, formaldehyde, gum benzoin, imidazolidinyl urea, phenyl-mercuric acetate, poly aminopropyl biguanide, proply gallate, sorbic acid, cresol, chloroacetamide sodium benzoate, chloromethyl-methylisothiazolinone, chloromethyl-methylisothiazolon, chloromethyl-methylisothiazolinone benzalkonium chloride, an octylisothiazolinone benzimidazol-compound, chloromethyl-methylisothiazolinone octylisothiazolinone, o-phenylphenol benzisothiazolinone, o-phenylphenol benzisothiazolinone, benzisothiazolinone, an aliphatic amine of 2-thiopyridineoxide, benzoic acid, editic acid, phenolic acid, benzyl alcohol, isopropyl alcohol, benzenethonium chloride, bronopol, cetrimide, chlorohexidine, chlorobutanol, chlorocresol, phenol, phenoxyethanol, phenyl ethyl alcohol, phenylmercuric acetate, phenylmercuric borate, phenylmercuric nitrate, potassium sorbate, proplyene glycol, sodium benzoate, sodium propionate, thimerosol, pharmaceutically acceptable salts thereof, and combinations thereof.

35. The gel composition of claim 11, further comprising a preservative present up to about 0.3% (w/w) of the composition.

36. The gel composition of claim 11, further comprising a chelator.

37. The gel composition of claim 11, further comprising a chelator selected from the group of edetate disodium, Ethylenediaminetetraacetic acid (EDTA), Diethylenetriaminepentaacetic acid (DTPA), trans-1,2-diaminocyclohexanetetraacetic acid (CDTA), (ethylenedioxy) diethylenedinitrilotetraacetic acid (EGTA), and combinations thereof.

38. The gel composition of claim 11, further comprising a chelator present up to about 0.2% (w/w) of the composition.

39. A gel composition comprising:
(a) an antibiotic agent, an antifungal agent, or a combination thereof;
(b) a humactant;
(c) a preservative;
(d) a chelator;
(e) a base;
(f) a gelling agent; and
(g) a solvent;
the composition having a pH of about 5.50.

40. A gel composition consisting essentially of:
(a) an antibiotic agent, an antifungal agent, or a combination thereof;
(b) a humactant;
(c) a preservative;
(d) a chelator;
(e) a base;
(f) a gelling agent; and
(g) a solvent;
the composition having a pH of about 5.50.

41. A gel composition comprising:
(a) metronidazole;
(b) propylene glycol;
(c) methylparaben;
(d) propylparaben;
(e) edetate disodium;
(f) sodium hydroxide;
(g) carbomer 934P; and
(h) water;
the composition having a pH of about 5.50.

42. A gel composition consisting essentially of:
(a) metronidazole,
(b) propylene glycol;
(c) methylparaben;
(d) propylparaben;
(e) edetate disodium;
(f) sodium hydroxide;
(g) carbomer 934P; and
(h) water in about 93.85% (w/w);
the composition having a pH of about 5.50.

43. A gel composition comprising:
(a) metronidazole in about 0.75% (w/w),
(b) propylene glycol in about 3.0% (w/w);
(c) methylparaben in about 0.08% (w/w);
(d) propylparaben in about 0.02% (w/w);
(e) edetate disodium in about 0.05% (w/w);
(f) sodium hydroxide in about 0.25% (w/w);
(g) carbomer 934P in about 2.0% (w/w); and
(h) water in about 93.85% (w/w);
the composition having a pH of about 5.50.

44. A gel composition consisting essentially of:
(a) metronidazole in about 0.75% (w/w),
(b) propylene glycol in about 3.0% (w/w);
(c) methylparaben in about 0.08% (w/w);
(d) propylparaben in about 0.02% (w/w);
(e) edetate disodium in about 0.05% (w/w);
(f) sodium hydroxide in about 0.25% (w/w);
(g) carbomer 934P in about 2.0% (w/w); and
(h) water in about 93.85% (w/w);
the composition having a pH of about 5.50.

45. A method for inhibiting a microorganism, the method comprising contacting the microorganism with an effective amount of the composition of claim 1 for a period of time effective to inhibit the microorganism.

46. The method of claim 45, wherein the microorganism is a fungus.

47. The method of claim 45, wherein the microorganism is a bacterium.

48. The method of claim 45, wherein the microorganism is a yeast.

49. The method of claim 45, wherein the microorganism is a mold.

50. A method for treating bacterial vaginosis in a human patient which comprises intravaginal administration to a patient in need of such treatment an effective amount of the composition of claim 1 wherein the composition is introduced into the vagina at least once a day for a time period of at least one day.

51. The method of claim 50, wherein said introducing is carried out about one to about three times daily

52. The method of claim 50, wherein said introducing is carried out over a time period of about three to about ten days.

53. The method of claim 50, wherein said introducing is carried out over a time period of about three to about ten days, consecutively.
